(19) United States
(12) Patent Application Publication
(10) Pub. No.: US 2002/0019616 A1
(43) Pub. Date: Feb. 14, 2002

(54) Elastomeric laminate with film and strands suitable for a nonwoven garment

(76) Inventor: Oomman Painumoottil Thomas, Alpharetta, GA (US)

Correspondence Address: Pauley Petersen Kinne & Fejer, 2800 W. Higgins Road, Suite 365, Hoffman Estates, IL, 60195, US

(21) Appl. No.: 09/855,194

(22) Filed: May 14, 2001

Related U.S. Application Data
(60) Provisional application No. 60/204,323, filed on May 15, 2000.

(51) Int. Cl. A61F 13/49; B32B 5/12; B32B 25/02; B32B 25/10; B32B 31/30

(52) U.S. Cl. 604/373; 428/114; 428/299.7; 264/171.1; 264/210.1; 264/210.8; 264/342 RE; 156/167; 156/178; 156/179; 156/229

(57) ABSTRACT

An elastic laminate suitable for use with a nonwoven garment has an elastic film with elastic strands placed thereon. The laminate is particularly suitable for combining with facing materials when making “targeted elastic materials” for placement into the structure of the nonwoven garment. A disposable garment may then utilize the elastic laminate, or the targeted elastic material made therefrom, to include an area of elasticized gathering under tension to better conform to the body of the wearer.

### FIELD OF THE INVENTION

This invention relates to an elastomeric laminate suitable for use with a garment, such as a nonwoven pant garment, e.g., a diaper or training pants.

### BACKGROUND OF THE INVENTION

Garments, including pant-like absorbent garments, medical garments, and other products, are commonly made with an elastic band adjacent at least one of the garment openings. A pant-like garment, for instance, may have an elastic band adjacent the waist opening, each of the two leg openings, or all three of the openings. The elastic bands are intended to fit snugly around a wearer's body to serve as gaskets, which prevent or reduce leakage of waste materials from inside the garment. Elastic bands have also been employed in leg flaps that provide further leakage protection in pant-like garments, and in other auxiliary gasketing applications.

In conventional garments, the primary material for the garment is manufactured and assembled separately from the elastic bands. Following their separate manufacture, the elastic bands are attached to the primary material at some stage during manufacture of the garment by sewing, ultrasonic welding, thermal bonding, adhesive bonding, or the like. In the resulting product, the user can often see the elastic band as a distinct entity attached to the garment.

Because of competition, there is an incentive to reduce both material and manufacturing costs associated with garments, without sacrificing performance and quality. However, this should be accomplished without compromising the gasketing characteristics around the openings in the garment. Conventional elastic bands can be relatively expensive to incorporate into garments, because of the current need for separate manufacture and attachment of the bands.

On the other hand, strands of elastic material integrated into the fabric, and especially nonwoven fabric, in an effort to obviate the separate bands may present problems with delamination of the elastic from the surrounding fabric, especially as the elastic grows in diameter to provide higher tension areas. Problems may also include appearance and performance problems associated with irregularity of placement of the strands during high speed manufacture and additional appearance and performance problems associated with post manufacturing processes such as cutting the integrated-strand fabric which may expose the non-uniform strand placement or cause retraction or slippage of the strands within the nonwoven fabric.

### SUMMARY OF THE INVENTION

The present invention is directed to an elastic laminate for a garment having one or more garment openings for the wearer's waist, legs, arms, and the like. The elastic laminate may then be combined with additional material, such as facing sheets, to make a single composite material, sometimes referred to as a targeted elastic material (“TEM”) having a targeted elastic zone which may be aligned with the garment opening or openings. The TEM may have a substantially homogeneous appearance, and does not have a separately manufactured elastic band attached to it. Yet the TEM may have different elastic properties at different regions, and exhibits greater elastic tension and/or greater elongation in a region aligned with, and in the vicinity of, at least one garment opening. The elastic laminate provides better adhesion to its surrounding fabric, a more cloth-like look, eliminates elastic strand slippage caused by usage of thicker elastic fibers; provides processing advantages such as eliminating custom extrusion dies, and provides better post processing appearance, such as when cutting to form smaller strips of elastic material; and will give stretch and better stress relaxation performance as a result of the lamination. Furthermore, a garment can be produced according to the present invention without the use of a separately manufactured, separately attached elastic band, and is easier and less expensive to manufacture than a conventional garment having one or more elastic bands at the opening.

With the foregoing in mind, it is a feature and advantage of the invention to provide an elastomeric material for use with a garment having a targeted elastic region aligned with, and in the vicinity of at least one garment opening, while eliminating the separate manufacture and attachment of an elastic band.

It is also a feature and advantage of the invention to provide various techniques for providing an elastic material which may have its elasticity varied by manipulation of its individual components' basis weight or physical structure.

The elastomeric material of the present invention is, at a first level, a combination, or composite, of elastomeric film and strands which has superior adherence to overlaying nonwoven fiber webs to which it is applied. At a second level the present invention may be considered to be the incorporation of the composite elastomeric into the web of fibrous material used to make the precursor garments. At another level, the present invention may be considered to be the incorporation of the fibrous material with the integral composite elastomeric into the finished garment.

These and other features and advantages will become further apparent from the following detailed description of the presently preferred embodiments, read in conjunction with the drawings.

### BRIEF DESCRIPTION OF THE DRAWINGS

FIG. 1 illustrates a perspective view of a pant-like absorbent garment in accordance with the invention, having targeted elastic gasket regions aligned with, and in the vicinity of garment openings;

FIG. 2 is a plan view of the garment shown in FIG. 1, showing the side facing away from the wearer;

FIG. 3 is a plan view of the garment shown in FIG. 1, showing the side facing the wearer;

FIGS. 4-8 illustrate representative examples of the elastic laminate materials of the present invention;

FIG. 9 illustrates a representative process for making the elastic laminate and targeted elastic materials (TEM) useful for making garments in accordance with the invention;

FIG. 10A shows one exemplary adhesive spray pattern in which the adhesive has been applied to the elastic filaments with attenuation in the cross direction;

FIG. 10B shows a second exemplary adhesive spray pattern;

FIG. 10C illustrates a third exemplary adhesive spray pattern;

FIG. 10D shows an exemplary bond angle in one exemplary adhesive spray pattern;

FIG. 11 illustrates the bonding pattern and method of calculating the number of bonds per unit length on elastic strands or filaments;

FIG. 12A shows a fourth exemplary adhesive spray pattern in a swirled-type of configuration;

FIG. 12B shows a fifth exemplary adhesive spray pattern that is more randomized and which provides a large percentage of adhesive lines in a perpendicular orientation to the elastic filaments;

FIG. 12C illustrates a sixth exemplary adhesive spray pattern having attenuation of adhesive lines in the cross-machine direction;

FIG. 12D shows a seventh exemplary adhesive spray pattern that resembles a “chain-link fence;”

FIG. 13 illustrates a side view of an extruder die in relation to a first roller, as may be used with the apparatus of FIG. 9;

FIG. 14 illustrates stress relaxation behavior of TE and non-TE materials at body temperature;

FIG. 15 illustrates hysteresis behavior of TE and non-TE materials;

FIG. 16 illustrates stretch-to-stop behavior of TE and non-TE materials; and

FIG. 17 is a schematic view of another process for making the elastic laminate and targeted elastic materials (TEM) useful for making garments in accordance with the invention.

### DEFINITIONS

The terms “elastic” and “elastomeric” are used interchangeably to mean a material that is generally capable of recovering its shape after deformation when the deforming force is removed. Specifically, as used herein, elastic or elastomeric is meant to be that property of any material which upon application of a biasing force, permits that material to be stretchable to a stretched biased length which is at least about 50 percent greater than its relaxed unbiased length, and that will cause the material to recover at least 40 percent of its elongation upon release of the stretching force. A hypothetical example which would satisfy this definition of an elastomeric material would be a one (1) inch sample of a material which is elongatable to at least 1.50 inches and which, upon being elongated to 1.50 inches and released, will recover to a length of less than 1.30 inches. Many elastic materials may be stretched by much more than 50 percent of their relaxed length, and many of these will recover to substantially their original relaxed length upon release of the stretching force.

The term “inelastic” refers to materials that are not elastic.

The term “gasket” or “gasket region” refers to a region of a garment which exhibits a moderate level of elastic tension against a wearer's body during use, and which restricts the flow of liquid and other material through a garment opening between the inside and outside of the garment. The term “fluid sealing gasket” is synonymous with these terms.

The term “targeted elastic regions” refers to isolated, often relatively narrow regions or zones in a single composite material or layer, which have greater elastic tension and/or elongation than adjacent or surrounding regions.

The term “vertical filament stretch-bonded laminate” or “VF SBL” refers to a stretch-bonded laminate made using a continuous vertical filament process, as described herein.

The term “elastic tension” refers to the amount of force per unit width required to stretch an elastic material (or a selected zone thereof) to a given percent elongation.

The term “elongation” refers to the capability of an elastic material to be stretched a certain distance, such that greater elongation refers to an elastic material capable of being stretched a greater distance than an elastic material having lower elongation.

The term “low tension zone” or “lower tension zone” refers to a zone or region in a stretch-bonded laminate material having one or more filaments with low elastic tension characteristics relative to the filament(s) of a high tension zone, when a stretching or biasing force is applied to the stretch-bonded laminate material. Thus, when a biasing force is applied to the material, the low tension zone will stretch more easily than the high tension zone. At 50% elongation of the fabric, the high tension zone may exhibit elastic tension at least 10% greater, suitably at least 50% greater, desirably about 100-800% greater, alternatively about 150-300% greater than the low tension zone.

The term “high tension zone” or “higher tension zone” refers to a zone or region in a stretch-bonded laminate material having one or more filaments with high elastic tension characteristics relative to the filament(s) of a low tension zone, when a stretching or biasing force is applied to the stretch-bonded laminate material. Thus, when a biasing force is applied to the material, the high tension zone will stretch less easily than the low tension zone. The terms “high tension zone” and “low tension zone” are relative, and the material may have multiple zones of different tensions.

The term “nonwoven fabric or web” means a web having a structure of individual fibers or filaments which are interlaid, but not in an identifiable manner as in a knitted fabric. The terms “fiber” and “filament” are used herein interchangeably. Nonwoven fabrics or webs have been formed from many processes such as, for example, meltblowing processes, spunbonding processes, air laying processes, and bonded carded web processes. The basis weight of nonwoven fabrics is usually expressed in ounces of material per square yard (osy) or grams per square meter (gsm) and the fiber diameters are usually expressed in microns. (Note that to convert from osy to gsm, multiply osy by 33.91.)

As used herein, the term “spunbond fibers” refers to small diameter fibers which are formed by extruding molten thermoplastic material as filaments from a plurality of fine, usually circular capillaries of a spinneret as taught, for example, by U.S. Pat. No. 4,340,563 to Appel et al. and U.S. Pat. No. 3,802,817 to Matsuki et al.

As used herein, the term “meltblown fibers” refers to fibers formed by extruding a molten thermoplastic material through a plurality of fine, usually circular, die capillaries as molten threads or filaments into converging high velocity gas streams (for example, airstreams) which attenuate the filaments of molten thermoplastic material to reduce their diameter, which may be to microfiber diameter. Such a process is disclosed, for example, by U.S. Pat. No. 3,849,241 to Butin.

As used herein, the term “microfibers” refers to small diameter fibers having an average diameter not greater than about 75 microns, for example, having an average diameter of from about 0.5 microns to about 50 microns, or more particularly, having an average diameter of from about 2 microns to about 40 microns.

The term “polymer” generally includes but is not limited to, homopolymers, copolymers, including block, graft, random and alternating copolymers, terpolymers, etc., and blends and modifications thereof. Furthermore, unless otherwise specifically limited, the term “polymer” shall include all possible geometrical configurations of the material. These configurations include, but are not limited to isotactic, syndiotactic and atactic symmetries.

The term “substantially continuous filaments or fibers” refers to filaments or fibers prepared by extrusion from a spinnerette, including without limitation spunbonded and meltblown fibers, which are not cut from their original length prior to being formed into a nonwoven web or fabric. Substantially continuous filaments or fibers may have lengths ranging from greater than about 15 cm to more than one meter; and up to the length of the nonwoven web or fabric being formed. The definition of “substantially continuous filaments or fibers” includes those which are not cut prior to being formed into a nonwoven web or fabric, but which are later cut when the nonwoven web or fabric is cut.

The term “recover” or “retract” relates to a contraction of a stretched material upon termination of a biasing force following stretching of the material by application of the biasing force.

The term “stretch to stop” or “STS” indicates the percentage of elongation of an elastic material when placed under a tensile load of 2000 grams.

The term “garment” includes personal care garments, medical garments, and the like. The term “disposable garment” includes garments which are typically disposed of after 1-5 uses. The term “personal care garment” includes diapers, training pants, swim wear, absorbent underpants, adult incontinence products, feminine hygiene products, and the like. The term “medical garment” includes medical (i.e., protective and/or surgical) gowns, caps, gloves, drapes, face masks, and the like. The term “industrial workwear garment” includes laboratory coats, coveralls, and the like.

“Inward” and “outward” refer to positions relative to the center of an article, and particularly transversely and/or longitudinally closer to or away from the longitudinal and transverse center of the article, and are analogous to proximal and distal.

The term “film” refers to an article of manufacture whose width exceeds its height and provides the requisite functional advantages and structure necessary to accomplish the claimed invention.

The term “strand” refers to an article of manufacture whose width is less than a film and is suitable for securement to a film according to the present invention.

The term “series” refers to a set including one or more elements.

The term “thermoplastic” describes a material that softens when exposed to heat and which substantially returns to a nonsoftened condition when cooled to room temperature.

The term “thermoset” describes a material that is capable of becoming permanently cross-linked.

With respect to the term “cross-link,” while linear molecules are important, they are not the only type of polymer molecules possible. Branched and cross-linked polymer molecules also play an important role in the structure and properties of polymers. When additional polymer chains emerge from the backbone of a linear polymer chain, it is said to be branched. Branching is introduced intentionally by adding monomers with the capability to act as a branch. The amount of branching introduced must be specified to characterize a polymer molecule completely. The branching points are referred to as junction points. When the concentration of the junction points is low, the molecules may be characterized by the number of chain ends. For example., two linear molecules have four chain ends. If one of this linear molecule is attached to the middle of the other linear molecule the resulting structure looks like a “T”. The total number of chain ends of this “T” molecule is three. Addition of another “T” to the end of another “T” will result in four chain ends. This process can be continued until a critical concentration of the resulting junction points is reached. Further coupling of the chain ends leads to a transition that transforms a solvent soluble, and a thermally processable branched polymer to an infusible and insoluble polymer mass. The number of junction points in such a mass becomes so high that the polymer molecule is theoretically considered to be one giant molecule that has a three-dimensional network structure. When this condition is achieved it is said to be cross-linked. Polymer molecules can be cross-linked in several ways, by changing the chemistry or by irradiating it with high energy beams such as UV, gamma ray, e-beam, etc. Some examples of chemical cross-linking are: 1) natural rubber, cis-1,4-polyisoprene, cross-linked with sulfur. This was discovered by Goodyear in 1839. This reaction is also known as vulcanization; 2) vinyl polymers cross-linked with divinyl monomers, for example polystyrene polymerized in the presence of divinyl benzene, 3) condensation polymers prepared from monomer of functionality greater than two, for example polyester formed with some glycerol or tricarboxylic acid, and 4) polysilicones cross-linked by reaction of benzoyl peroxide. An example of cross-linking by high energy electron beam is the cross-linking of polyethylene by radiation.

### DETAILED DESCRIPTION OF THE PRESENTLY PREFERRED EMBODIMENTS

The principles of this invention can be applied to a wide variety of garments, including disposable garments, having a targeted elastic zone in the vicinity of at least one garment opening. Examples include diapers, training pants, certain feminine hygiene products, adult incontinence products, other personal care or medical garments, and the like. For ease of explanation, the following description is in terms of a refastenable child training pant having elastic material used for containment flaps and a waist dam.

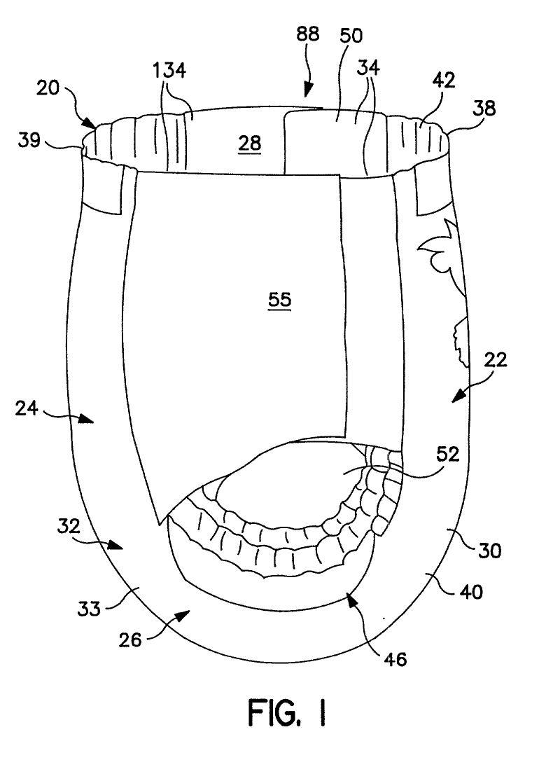

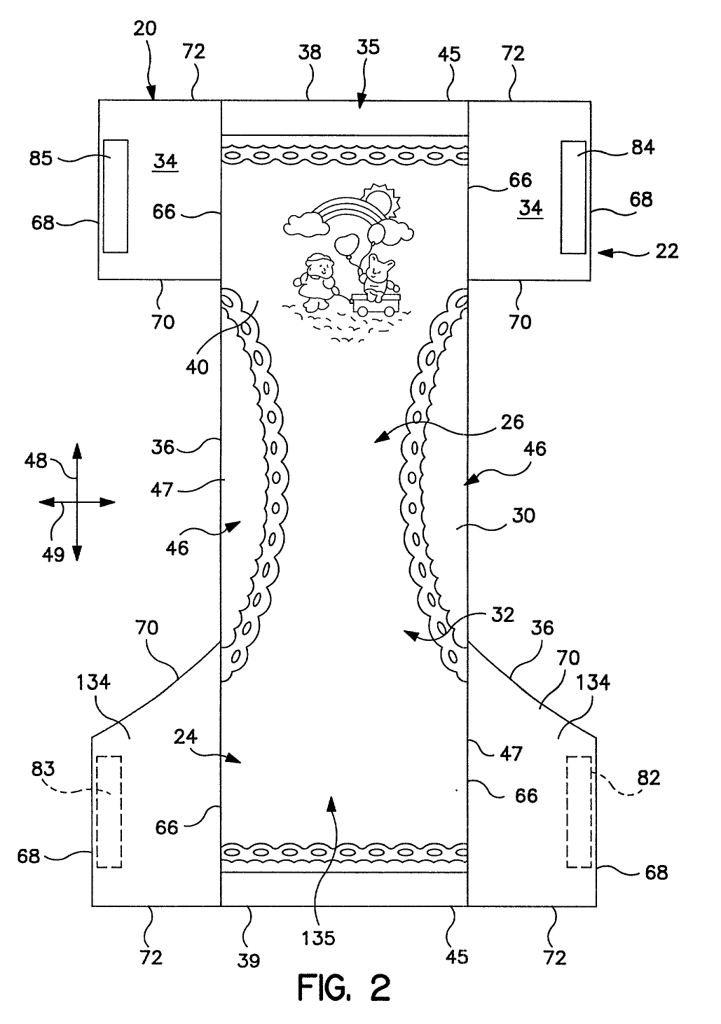

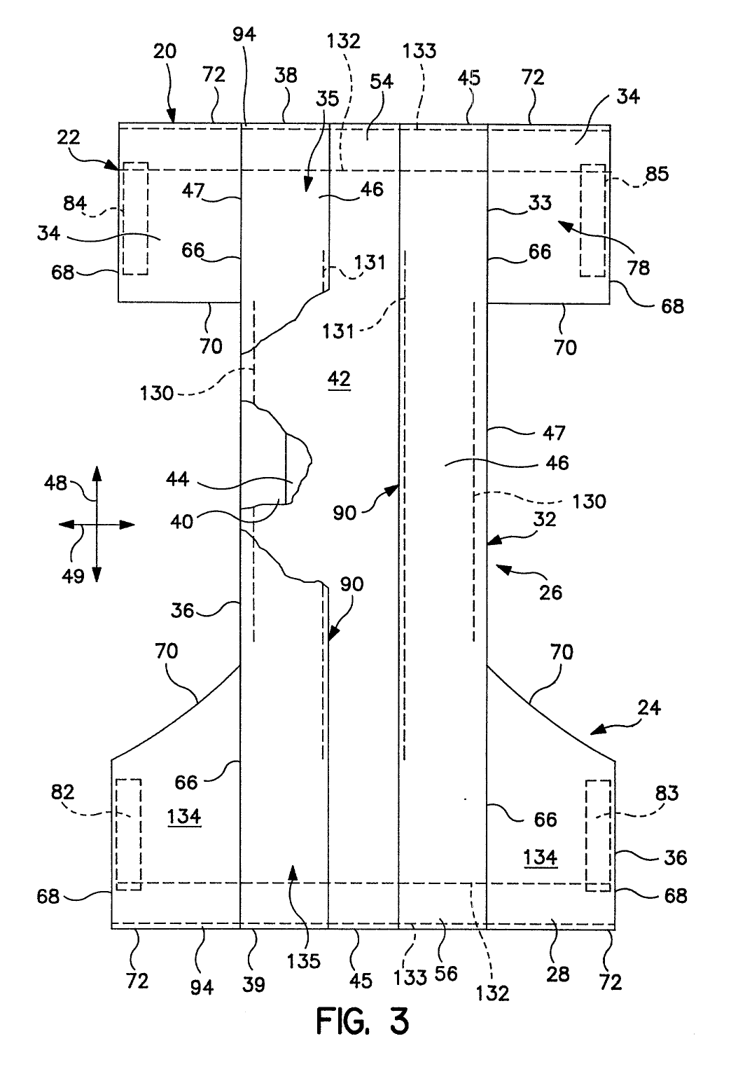

Referring to FIG. 1, a disposable absorbent garment 20, such as a child training pant, includes an absorbent chassis 32 and a fastening system 88. The absorbent chassis 32 defines a front waist region 22, a back waist region 24, a crotch region 26 interconnecting the front and back waist regions, an inner surface 28 which is configured to contact the wearer, and an outer surface 30 opposite the inner surface which is configured to contact the wearer's clothing. With additional reference to FIGS. 2 and 3, the absorbent chassis 32 also defines a pair of transversely opposed side edges 36 and a pair of longitudinally opposed waist edges, which are designated front waist edge 38 and back waist edge 39. The front waist region 22 is contiguous with the front waist edge 38, and the back waist region 24 is contiguous with the back waist edge 39. The chassis 32 defines waist opening 50 and two opposing leg openings 52.

The illustrated absorbent chassis 32 comprises a rectangular absorbent composite structure 33, a pair of transversely opposed front side panels 34, and a pair of transversely opposed back side panels 134. The composite structure 33 and side panels 34 and 134 may be integrally formed or comprise two or more separate elements, as shown in FIG. 1. The illustrated composite structure 33 comprises an outer cover 40, a bodyside liner 42 (FIGS. 1 and 3) which is connected to the outer cover in a superposed relation, an absorbent assembly 44 (FIG. 3) which is located between the outer cover and the bodyside liner, and a pair of containment flaps 46 (FIG. 3). The rectangular composite structure 33 has opposite linear end edges 45 that form portions of the front and back waist edges 38 and 39, and opposite linear side edges 47 that form portions of the side edges 36 of the absorbent chassis 32 (FIGS. 2 and 3). For reference, arrows 48 and 49 depicting the orientation of the longitudinal axis and the transverse axis, respectively, of the training pant 20 are illustrated in FIGS. 2 and 3.

The front waist region 22 of the absorbent chassis 32 includes the transversely opposed front side panels 34 and a front center panel 35 (FIGS. 2 and 3) positioned between and interconnecting the side panels. The back waist region 24 of the absorbent chassis 32 includes the transversely opposed back side panels 134 and a back center panel 135 (FIGS. 2 and 3) positioned between and interconnecting the side panels. The waist edges 38 and 39 of the absorbent chassis 32 are configured to encircle the waist of the wearer when worn and provide the waist opening 50 which defines a waist perimeter dimension. Portions of the transversely opposed side edges 36 in the crotch region 26 generally define the leg openings 52.

In the embodiment shown in FIG. 1, the front and back side panels 34 and 134 are fastened together by fastening system 88 to form collective side panels 55 (with each collective side panel 55 including a front side panel 34 and back side panel 134). The fastening system 88 may include a plurality of fastener tabs 82, 83, 84 and 85, which can be known hook-and-loop fastener members. It will be appreciated that any number of side panel configurations maybe utilized in the context of the present invention.

The illustrated side panels 34 and 134, in FIGS. 2 and 3, each define a distal edge 68 that is spaced from the attachment line 66, a leg end edge 70 disposed toward the longitudinal center of the training pant 20, and a waist end edge 72 disposed toward a longitudinal end of the training pant. The leg end edge 70 and waist end edge 72 extend from the side edges 47 of the composite structure 33 to the distal edges 68. The leg end edges 70 of the side panels 34 and 134 form part of the side edges 36 of the absorbent chassis 32. In the back waist region 24, the leg end edges 70 are desirably although not necessarily angled relative to the transverse axis 49 to provide greater coverage toward the back of the pant as compared to the front of the pant. The waist end edges 72 are desirably parallel to the transverse axis 49. The waist end edges 72 of the front side panels 34 form part of the front waist edge 38 of the absorbent chassis 32, and the waist end edges 72 of the back side panels 134 form part of the back waist edge 39 of the absorbent chassis.

Referring to FIGS. 1-3, in accordance with the invention, the containment flaps 46, desirably continuous with the chassis 32, each include a targeted elastic material (TEM) including an elasticized, low tension and/or high stretch zone 130 in the vicinity of (and aligned with) leg openings 52, and a narrow, band-like high tension and/or low stretch zone 131 in the vicinity of (and aligned with) the unattached, gasket-like edges 90 of the containment flaps 46 thereby creating a gasket at the gasket-like edges 90 of the containment flaps 46 (FIG. 3). The containment flaps 46 can be separate, attached pieces (as shown in FIGS. 1 and 2), or can be an extension of the outer cover 40, as shown in FIG. 3. The dotted lines in FIG. 3 indicate the boundaries between the low tension and/or high stretch zone 130 and the high tension and/or low stretch zone 131, which boundaries are not visible to an observer. The low tension and/or high stretch zone 130 and the high tension and/or low stretch zone 131 are suitably spaced apart, as shown in FIG. 3. From the standpoint of the observer, the TEM forming the containment flaps 46 appears as a homogeneous, integrated material.

The high tension and/or low stretch zone 131 exhibits greater elastic tension and/or elongation than the low tension and/or high stretch zone 130 of the containment flaps 46, without requiring the use of separately manufactured and attached elastic materials. Furthermore, desired spacing between the high tension and/or low stretch zone 131 and the low tension and/or high stretch zone 130 allows the zones 131 and 130 to stretch independently of one another so as not to constrain elongation capacity of either zone 131 and 130.

To further enhance containment and/or absorption of body exudates, the training pant 20 desirably includes a waist dam having a front waist dam portion 54 and a rear waist dam portion 56 (FIG. 3) of a high tension and/or low stretch zone 133 in the vicinity of (and aligned with) the waist edges 38 and 39. The waist dam portions 54 and 56 can be separate, attached pieces, or can be extensions of the outer cover 40, as shown in FIG. 3. From the standpoint of the observer, the TEM forming the waist dam portions 54 and 56 appears as a homogeneous, integrated material.

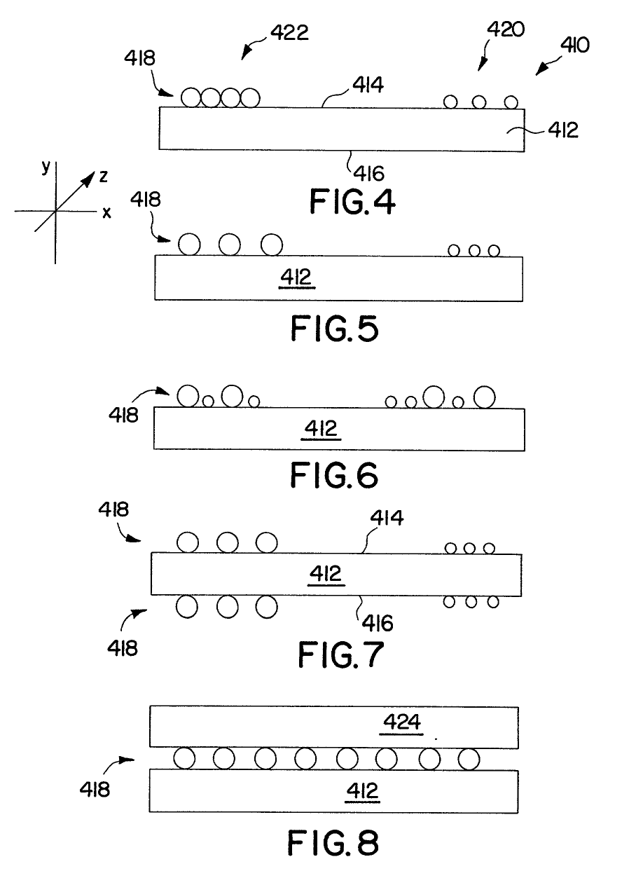

The containment flaps 46 and the waist dam portions 54 and 56 are manufactured from a targeted elastic material. Various embodiments of targeted elastic materials may include the elastic laminate materials shown in FIGS. 4-8.

As seen in FIG. 4, an elastomeric laminate 410 comprises an elastomeric film 412 having a first major surface 414 and a second major surface 416. Secured to the first major surface 414 are strands 418 of elastomeric material. The longitudinal axes of the film 412 and the strands, collectively 418, run in the same direction, which in FIGS. 4-8 is the indicated Z direction going into the illustration. The elastomeric strands 418 are suitably but not necessarily secured to the film 412 by a combination of tackifiers within the elastomeric compositions and an application of melt sprayed adhesive on the film's major surface. The right side 420 and left side 422 of the film 412 may have differential spacing among their respectively grouped strands which can impart a different level of tension between the two areas. It will be appreciated that the strands may be laid out periodically, non-periodically, and in various spacings, groupings, sizes, and compositions of elastic material according to the effect desired from the elastic laminate and the use to which it is put. For example, FIG. 5 illustrates unequal sized elastomeric strands 418 with the left side grouping being of larger diameter and thus of higher tension than the smaller diameter right side grouping. While referred to as being of different diameter, it will be appreciated that the elastomeric strands 418 need not be circular in cross-section within the context of the present invention. FIG. 6 illustrates that the strands of different size may be intermingled within groupings in regular or irregular patterns. FIG. 7 illustrates that various strands 418 may be secured to both of the first and second major surfaces 414, 416 respectively, of the film 412. FIG. 8 illustrates that the laminate of the film 412 and strands 418 may have an additional film 424 secured to the strands 418 thereby sandwiching the strands 418 between the first, or original, film 412 and the second film 424. All of the above techniques as well as the basis weight and physical structure, e.g. strand-like, film-like or meltblown structures may be utilized, in conjunction with the chemical compositions of the laminate elements to vary the elastic tension of the laminate as a whole. Also, the tension of different portions of the film 412 can be varied from one another, and in addition, the tension among the strands 418 can vary from one another as well. Furthermore, rather than a film 412, a sheet of netting or nonwoven may instead be used as the substrate for attaching the strands 418.

Materials suitable for use in preparing elastomeric films and strands include diblock, triblock, tetrablock or other multi-block elastomeric copolymers such as olefinic copolymers, including styrene-isoprene-styrene, styrene-butadiene-styrene, styrene-ethylene/butylene-styrene, or styrene-ethylene/propylene-styrene, which may be obtained from the Shell Chemical Company, under the trade designation KRATON® elastomeric resin; polyurethanes, including those available from E. I. Du Pont de Nemours Co., under the trade name LYCRA® polyurethane; polyamides, including polyether block amides available from Ato Chemical Company, under the trade name PEBAX® polyether block amide; polyesters, such as those available from E. I. Du Pont de Nemours Co., under the trade name HYTREL® polyester; and single-site or metallocene-catalyzed polyolefins having density less than about 0.89 grams/cc, available from Dow Chemical Co. under the trade name AFFINITY™.

A number of block copolymers can also be used to prepare elastomeric strands and films 418, 412 useful in this invention. Such block copolymers generally comprise an elastomeric midblock portion B and a thermoplastic endblock portion A. The block copolymers may also be thermoplastic in the sense that they can be melted, formed, and resolidified several times with little or no change in physical properties (assuming a minimum of oxidative degradation). Alternatively, the elastomeric strands 418 and/or films 412 can be made of a polymer that is not thermally processable, such as LYCRA® spandex, available from E. I. Du Pont de Nemours Co., or cross-linked natural rubber in film or fiber form. Thermoset polymers and polymers such as spandex, unlike the thermoplastic polymers, once cross-linked cannot be thermally processed, but can be obtained on a spool or other form and can be stretched and applied to the film 412 or strands 418 in the same manner as thermoplastic polymers. As another alternative, the elastomeric strands 418 and/or films 412 can be made of a thermoset polymer, such as AFFINITY®, available from Dow Chemical Co., that can be processed like a thermoplastic, i.e. stretched and applied, and then treated with radiation, such as electron beam radiation, gamma radiation, or UV radiation to cross-link the polymer, or use polymers that have functionality built into them such that they can be moisture-cured to cross-link the polymer, thus resulting in a polymer with the enhanced mechanical properties of a thermoset.

Endblock portion A may comprise a poly(vinylarene), such as polystyrene. Midblock portion B may comprise a substantially amorphous polyolefin such as polyisoprene, ethylene/propylene polymers, ethylene/butylene polymers, polybutadiene, and the like, or mixtures thereof.

Suitable block copolymers useful in this invention include at least two substantially polystyrene endblock portions and at least one substantially ethylene/butylene mid-block portion. A commercially available example of such a linear block copolymer is available from the Shell Chemical Company under the trade designation KRATON® G1657 elastomeric resin. Another suitable elastomer is KRATON® G2740.

Elastic elements of the present invention may also contain blends of elastic and inelastic polymers, or of two or more elastic polymers, provided that the blend exhibits elastic properties. The strands are substantially continuous in length. The elastic strands may be circular but as previously mentioned, may also have other cross-sectional geometries such as elliptical, rectangular, triangular or multi-lobal. In one embodiment, one or more of the filaments may be in the form of elongated, rectangular strips produced from a film extrusion die having a plurality of slotted openings.

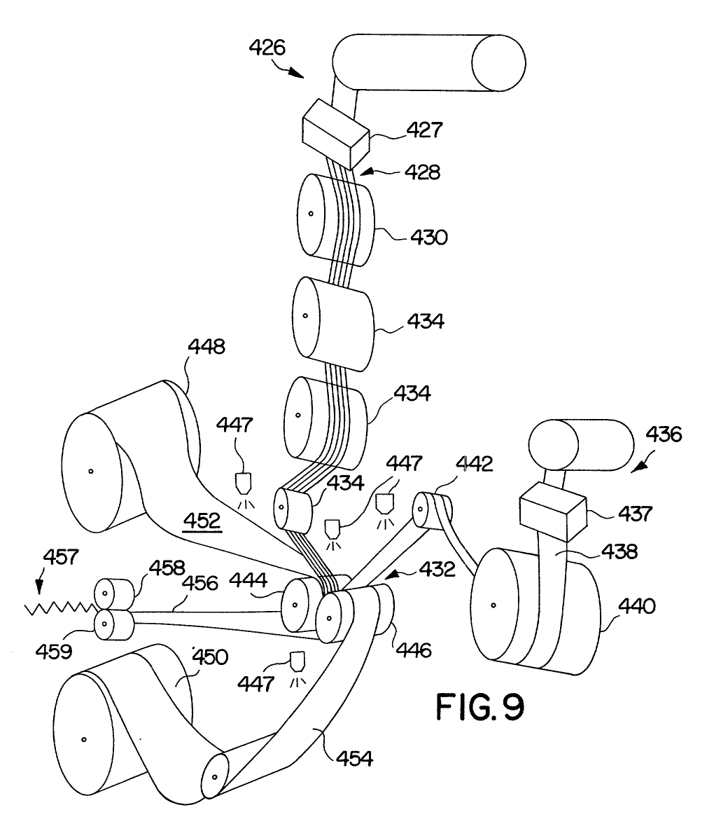

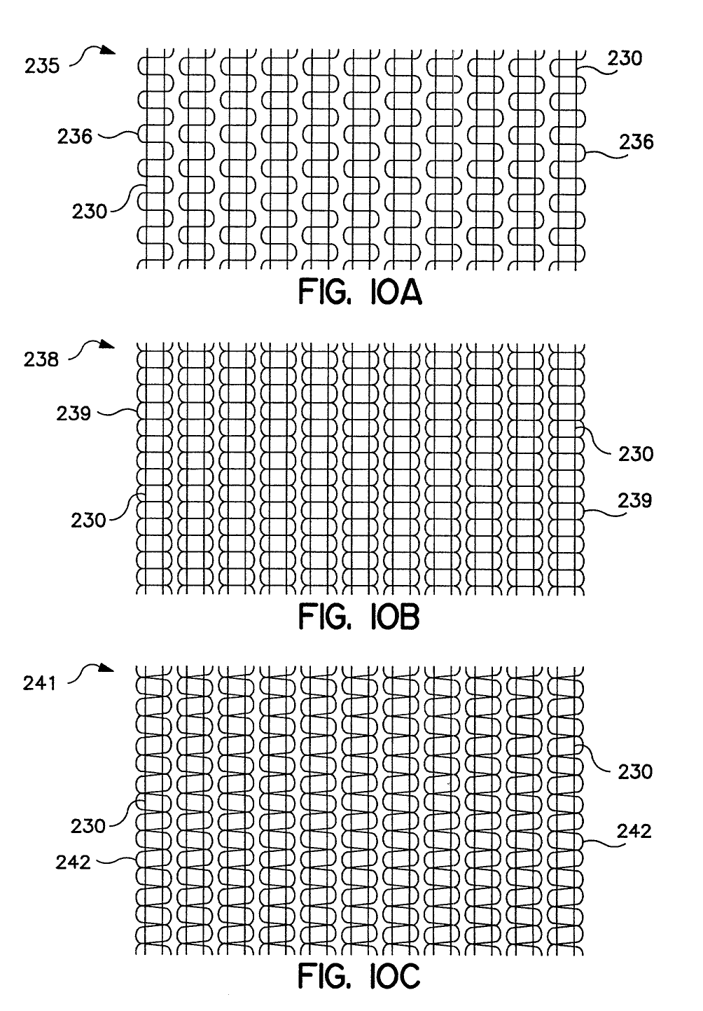

FIG. 9 illustrates a method and apparatus for making an elastic laminate according to FIGS. 4-7 and forming a targeted elastic material from the elastic laminate. The double filmed laminate of FIG. 8 would of course have another line added for forming the second film. While FIG. 9 illustrates a composite VF SBL process it will be appreciated that other processes consistent with the present invention may be utilized. A first extruder 426 produces strands of elastic material 428 through a filament die 427. The strands 428 are fed to a first chill roller 430 and stretched conveyed vertically towards a nip 432 by one or more first fly rollers, collectively 434, in the strand-producing line.

A second extruder 436 using a slotted film die 437 produces a film of elastic material 438, of e.g. about 7.5″ in width and ten (10) mils thickness, which is fed onto a second chill roller 440 and conveyed to one or more second fly rollers, collectively 442, towards the nip 432. The film 438 may be stretched down to about two inches width and thinned to about 2 mils by the second fly rollers 442 during its passage to the nip 432. The nip 432 is formed by opposing first and second nip rollers 444 and 446, respectively. The elastic laminate 410 (FIG. 4) is formed by securing the strands 428 to the film 438 in the nip 432 by heat, pressure, adhesives or combinations thereof. Adhesive sprayers, collectively 447, may be placed as desired on each material's path before entry into the nip.

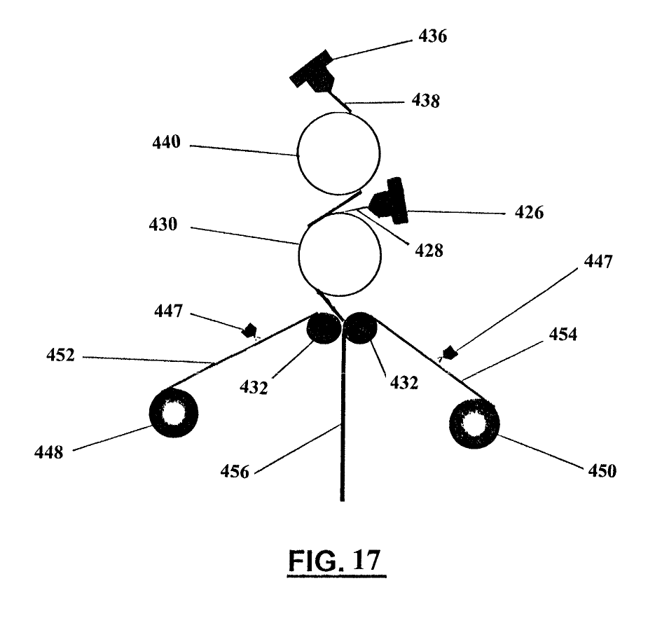

FIG. 17 illustrates a VF SBL process in which no fly rollers 434 are used. Instead, the film 438 is extruded onto chill roller 440. The strands 428 are extruded onto chill roller 430, where the strands 428 and the film 438 converge. The strands 428 and the film 438 are stretched between the chill rollers 430, 440 and the nip 432. Except for the lack of fly rollers 434, the processes of FIGS. 9 and 17 are similar. In either case, the strands 428 and the film 438 together are laminated between a first facing layer 452 and a second facing layer 454 at the nip 432.

FIG. 13 illustrates a side view of an extruder 15 in a canted position relative to the vertical axis of a roller 12. The 45° angle indicated on the Figure has been found to be one angle that produces an acceptable product and that allows the continuous filaments to mate with roller 12.

The die of each extruder 15 may be positioned with respect to the first roller 12 so that the continuous filaments 14 meet this first roller 12 at a predetermined angle 16. This strand extrusion geometry is particularly advantageous for depositing a melt extrudate onto a rotating roll or drum. An angled, or canted, orientation provides an opportunity for the filaments to emerge from the die at a right angle to the roll tangent point resulting in improved spinning, more efficient energy transfer, and generally longer die life. This improved configuration allows the filaments to emerge at an angle from the die and follow a relatively straight path to contact the tangent point on the roll surface. The angle 16 between the die exit of the extruder and the vertical axis (or the horizontal axis of the first roller, depending on which angle is measured) may be as little as a few degrees or as much as 90. For example, a 90° extrudate exit to roller angle could be achieved by positioning the extruder directly above the downstream edge of the first roller and having a side exit die tip on the extruder. Moreover, angles such as about 20°, about 35°, or about 45° away from vertical may be utilized. It has been found that, when utilizing a 12-filament/inch spinplate hole density, an approximately 45° angle (shown in FIG. 13) allows the system to operate effectively. The optimum angle, however, will vary as a function of extrudate exit velocity, roller speed, vertical distance from the die to the roller, and horizontal distance from the die centerline to the top dead center of the roller. Optimal performance can be achieved by employing various geometries to result in improved spinning efficiency and reduced filament breakage. In many cases, this results in potentially increased roll wrap resulting in more efficient energy transfer and longer die life due to reduced drag and shear of the extrudate as it leaves the capillaries of the extruder die and proceeds to the chilled roll.

In order to form the TEM 456, first and second rolls 448 and 450, respectively, of spunbond facing material, 452 and 454, are fed into the nip 432 on either side of the elastic strands 428 and film 438 and bonded accordingly. The spunbond facing material might also be made in situ rather than unrolled from previously-made rolls of material. While illustrated as having two lightweight gatherable spunbond facings, it will be appreciated that only one facing material, or various types of facing materials, may be used. The bonded TEM 456 is maintained in stretched condition by a pair of tensioning rollers 458, 459 downstream of the nip and then relaxed as at Ref. No. 457.

The facing layer or layers 452,454 may each include a nonwoven web, for example a spunbonded web or a meltblown web, a woven web, or a film. Facing materials may be formed using conventional processes, including the spunbond and meltblowing processes described in the “DEFINITIONS.” For example, facing materials 452, 454 may include a spunbonded web having a basis weight of about 0.1-4.0 osy, suitably 0.2-2.0 osy, desirably about 0.4-0.6 osy. The facing materials may include the same or similar materials or different materials.

The facing materials 452, 454 can be bonded to the elastomeric laminate 410 by using an adhesive, for example an elastomeric adhesive such as Findley H2525A, H2525 or H2096. Other bonding means well known to those having ordinary skill in the art may also be used to bond the facing materials 452,454 to the elastic laminate 410, including thermal bonding, ultrasonic bonding, mechanical stitching and the like.

Several patents describe various spray apparatuses and methods that may be utilized in supplying the meltspray adhesive to the outer facing(s) or, when desired, to the elastic laminate. For example, the following United States patents assigned to Illinois Tool Works, Inc. (“ITW”) are directed to various means of spraying or meltblowing fiberized hot melt adhesive onto a substrate: U.S. Pat. Nos. 5,882,573; 5,902,540; 5,904,298. These patents are incorporated herein in their entireties by reference thereto. The types of adhesive spray equipment disclosed in the aforementioned patents are generally efficient in applying the adhesive onto the nonwoven outer facings in the VFL process of this invention. In particular, ITW-brand Dynatec spray equipment, which is capable of applying about 3 gsm of adhesive at a run rate of about 1100 fpm, may be used in the melt-spray adhesive applications contemplated by the present inventive process.

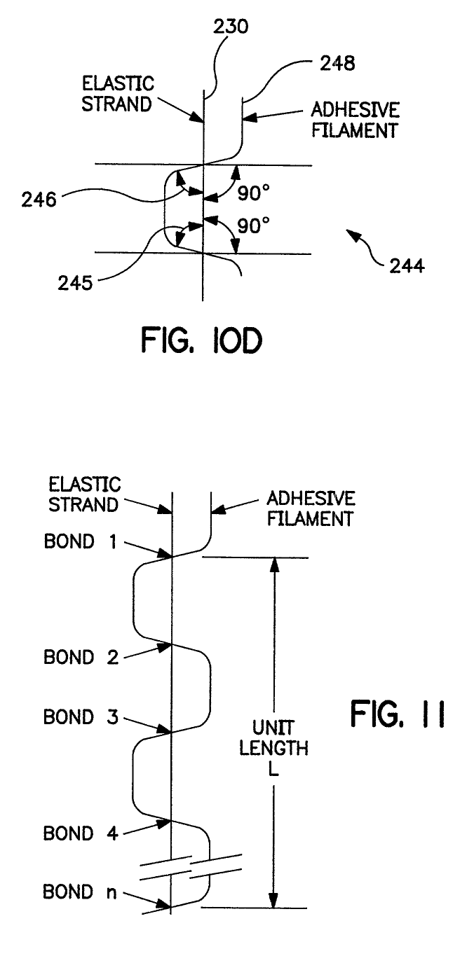

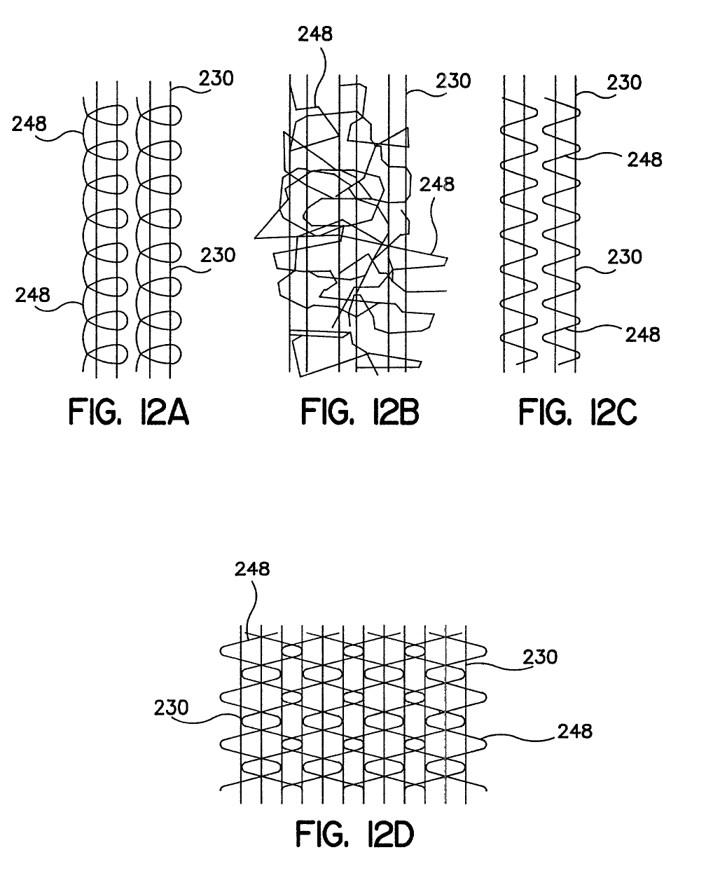

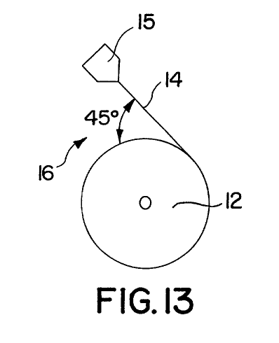

Several representative adhesive patterns are illustrated in FIGS. 10A through 12D. Applying an adhesive in a cross-machine pattern such as the ones shown in FIGS. 12C and 12D may result in certain adherence advantages. For example, because the elastic laminate is generally placed in the machine direction, or direction of processing, having the adhesive pattern orient to a large degree in the cross-machine direction provides multiple adhesives to elastic crossings per unit length. For this discussion, the elastic strands of the laminate of the present invention will be used for ease of illustration. It will be noted that the strands are oriented on the film of the laminate in the machine direction.

In addition, in many particular embodiments of the present invention, the adhesive component is applied to the surface of the nonwoven facing sheet, or layer, in discrete adhesive lines. The adhesive may be applied in various patterns so that the adhesive lines intersect the elastic filament lines to form various types of bonding networks which could include either adhesive-to-elastic bonds or adhesive-to-elastic bonds, adhesive-to-facing layer, and adhesive-to-adhesive bonds. These bonding networks may include a relatively large total number of adhesive-to-elastic and adhesive-to-adhesive bonds that provide the laminated article with increased strength, while utilizing minimal amounts of adhesive. Such enhancements are achieved by the use of adhesive sprayed onto the surface of the nonwoven in a predetermined and specific pattern. In most cases, a final product with less adhesive exhibits a reduction in undesirable stiffness, and is generally more flexible and soft than products having more adhesive.

Applying the adhesive in a pattern so that the adhesive lines are perpendicular or nearly perpendicular to the machine direction of the elastic components has been found particularly advantageous. A true 90° bond angle may not be possible in practice, but an average or mean bond angle that is as great as 50° or 60° will generally produce a suitable bond between the elastic laminate and the facing material. A conceptual illustration of these types of bond angles is shown in FIGS. 10D and 11. The adhesive-to-elastic bonds are formed where the lines of adhesive 248 and elastic strands 230 join or intersect.

The continuous adhesive filaments-to-elastic strand intersections are also controlled to a predetermined number of intersections per unit of elastic laminate length. By having such adhesive lines in a perpendicular orientation and optimizing the number of bonds per unit of elastic laminate length, the final bonded material, or TEM, can be produced with a minimal amount of adhesive and elastomeric material to provide desirable product characteristics at a lower cost.

If the adhesive-to-elastic bonds are too few in number or are too weak, then the elastic tension properties of the TEM may be compromised and the tension applied to the elastic may break the adhesive joints. In various known processes, the common remedy for this condition is to increase the number of bonding sites by either increasing the meltspray air pressure, or by slowing the bonding, or lamination, speed. As the meltspray air pressure is increased, the resulting adhesive fiber size is reduced, creating weaker bonds. Increasing the amount of adhesive used per unit area to create larger adhesive filaments can strengthen these weaker bonds, which usually increases the cost of the laminate. Lowering the lamination speed decreases machine productivity, negatively impacting product cost. The present invention, in part, may utilize an effective bonding pattern where the number of bond sites per length of elastic are prescribed and where the adhesive-to-elastic strand joints are generally perpendicular in orientation in order to provide maximum adhesive strength. This allows the TEM to be made at minimal cost by optimizing the adhesive and elastomer content to match the product needs.

As used herein, a “scrim” refers generally to a fabric or nonwoven web of material which may be elastic or inelastic, and having a machine direction (“MD”) oriented strand component along the path of product flow during manufacture and a cross-machine direction (“CD”) strand component across the width of the fabric.

FIG. 10A shows one exemplary scrim pattern useful in the present invention in which the adhesive has been applied to the elastic filaments with attenuation of the adhesive lines in the cross-machine direction. Scrim pattern 235 includes adhesive line 236 and elastic filaments 230. FIG. 10B illustrates another exemplary scrim pattern 238 having adhesive lines 239 applied to elastic strands 230. In this embodiment, it can be seen that the bond angle is very high, approaching 90° at the intersection between the adhesive and the elastic filaments. FIG. 10C illustrates still another scrim pattern 241 having adhesive lines 242 and continuous elastic strands 230.

As previously discussed, FIG. 10D illustrates the relatively high bond angle that may be employed in products produced according to the present invention. In particular, lay down angle 244 is shown as the angle formed by the adhesive line 248 and the elastic strand 230. Adhesive/elastic angle 246 and adhesive/elastic angle 245 are shown as being less than 90°.

FIG. 11 utilizes an exemplary bonding pattern to conceptually illustrate the measurement for determining the number of bonds per unit length on elastic strands or filaments. FIG. 12A shows another exemplary bonding pattern having the adhesive-to-adhesive bonding wherein a swirled type of configuration is employed. FIG. 12B illustrates a more randomized pattern wherein a large percentage of adhesive lines are in a perpendicular, or almost perpendicular, orientation to the elastic filaments. FIG. 12C is another exemplary embodiment of a bonding pattern having no adhesive-to-adhesive bonds, but numerous adhesive-to-elastic strand bonds. FIG. 12D illustrates another exemplary bonding pattern that has both adhesive-to-adhesive and adhesive-to-elastic strand bonds. The configuration shown in FIG. 12D is similar to the design of a chain-link fence.

### EXAMPLE 1

In an assembly known as the Vertical Filament Laminator (VFL), strands of an elastomeric polymer made up of 65.5% KRATON® G1730, 12% of a low molecular weight polyethylene wax, NA 601, and 22.5% of a pressure sensitive adhesive such as Regalrez™ of Hercules Inc., of Wilmington, Del., were extruded onto the top of a chill roll. The elastic strands were subsequently stretched successively through a series of rolls stacked in a vertical fashion, one on top of each other, under the chill roll and into a pair of nip rolls, i.e. rolls creating a nip. In the nip, the facing sheets and tackified elastic strands meet whereupon the strands are bonded to the facings, under pressure, to form a gathered but stretchable laminate. Alternatively, an external hot melt adhesive can be sprayed on the facing sheets, prior to entering the nip, in order to bond a non-tackified elastomer to the facing sheets.

In the VFL assembly, a film of the same elastomer was cast from a second extruder using a slotted film die at a width of 7.5 inches and approximately 10 mil thick adjacent to the strands. Because of the close proximity of the strands and film they make contact with each other at the initial cooling roller. The film width, initially at 7.5 inches, narrowed to 2 inches when passed over all the rolls, which were run at differential speed together with the strands. The film also thinned down to approximately 2 mils thickness in the final gathered laminate after passing through the nip. A difficulty was perceived in introducing the film and strands on top of the same chill roll together.

A second approach was adopted for the successful development of the film based banded or targeted elastic laminate by casting the film onto a separate chill roll using the slotted film die. The film was guided to the nip through one or more fly rolls and laminated together with the strands between the facings. In this construction, no attempt was made to separate the strands from the area in which film was present, the strand was laid just on top of the film. In other words, the strand lay down had no discontinuity. The stretch of the film and strands from their extruders had to be identical to produce a laminate with uniform gathering. To achieve a differential gathering of the elastic targeted zones, a differential stretch prior to bonding is recommended. The initial width and gap of the film die was adjusted to effect the width and thickness of the film in the final laminate. Alternatively, the forming distance (distance between the die and the chill roll), chill roll speed and polymer throughput can also be adjusted to change the dimensions of the film. It was observed during the processing that an increase in stretch of the elastomer to achieve a higher stretch to stop (STS) of 230-260%, when compared with a control material of 80-190% STS, results in delamination of the strands from the film. Use of excess adhesive in the elastomeric materials also results in reduction of stretch to stop of the laminate. Hence 1 gsm of a Findley 2096 adhesive was melt sprayed on the facing in addition to the tackifier present in the elastomer formulation which resulted in excellent adhesion and provided 230%+ elongation. Another observation made during the production of the elastic laminate was that the film chill roll temperature had to be around 25° C. to prevent the film from breaking. Of course, different formulations of laminate components may require different temperature controls.

### EXAMPLE 2

In this example, targeted elastic materials were tested in terms of stress relaxation at body temperature, a 3-cycle hysteresis test, and stress elongation. In the stress relaxation test, the samples tested included TE made up of a film including 65.5% KRATON® G1730, 12% of a low molecular weight polyethylene wax, NA 601, and 22.5% of a pressure sensitive adhesive such as Regalrez™, and filaments including 80% KRATON® G1730, 13% tackifier, and 7% wax, with the filaments overlaid on the film. Non-TE portions of a laminate were based solely on the filaments made up of 80% KRATON® G1730, 13% tackifier, and 7% wax. The control sample used in the stress relaxation test was a laminate based on LYCRA® spandex, available from E. I. Du Pont de Nemours Co., in a non-TE type laminate material construction. In the hysteresis test, the samples included a TE sample of film made up of 65.5% KRATON® G1730, 12% of a low molecular weight polyethylene wax, NA 601, and 22.5% of a pressure sensitive adhesive such as Regalrez™, together with filament made up of 85% KRATON™ G1730 and 15% wax, and a filament-based non-TE sample made up of 80% KRATON® G1730, 13% tackifier, and 7% wax. The control sample was the side panel material used in the PULL-UPS® Disposable Training Pant, based on KRATON® G 2760 polymer. In the stress elongation test, the samples included a TE sample of film made up of 65.5% KRATON® G1730, 12% of a low molecular weight polyethylene wax, NA 601, and 22.5% of a pressure sensitive adhesive such as Regalrez™, together with filament made up of 85% KRATON® G1730 and 15% wax, a filament-based non-TE sample of 80% KRATON® G1730, 13% tackifier, and 7% wax, and a control of filament-based non-TE sample of KRATON® 2760, which is the commercial side panel material used in PULL-UPS® Disposable Training Pants.

Stress Relaxation at Body Temperature

Stress relaxation of the elastomer at body temperature is used mainly for rating the dimensional stability of the material. Stress relaxation is defined as the force required to hold a given elongation constant over a period of time. Hence, it is a transient response which mimics personal care products in use. In this experiment, the load loss (stress relaxation) as a function of time was measured at body temperature. The rate of change of the property as a function of time was obtained by calculating the slope of a log-log regression of the load and time. In addition to the rate of loss as a function of time, the percentage of load loss was calculated from the knowledge of the initial and final loads. The duration of the experiment was matched with the time a product stays on the body in real use. A perfectly elastic material, such as a metal spring, for instance, is expected to give a value of zero for both slope and load loss.

In the stress relaxation characterization, a 3-inch width of the laminate specimen was used for the test. Samples were tested in a Sintech mechanical test frame in an environmental chamber at 100° F. (38° C.). An initial 3-inch grip-to-grip distance was displaced to a final 4.5 inches (50% elongation) at a cross-head displacement speed of 20 inches/minute. The load loss as a function of time was then acquired over a period of 12 hours using the Testworks data acquisition capability of the MTS Sintech test equipment.

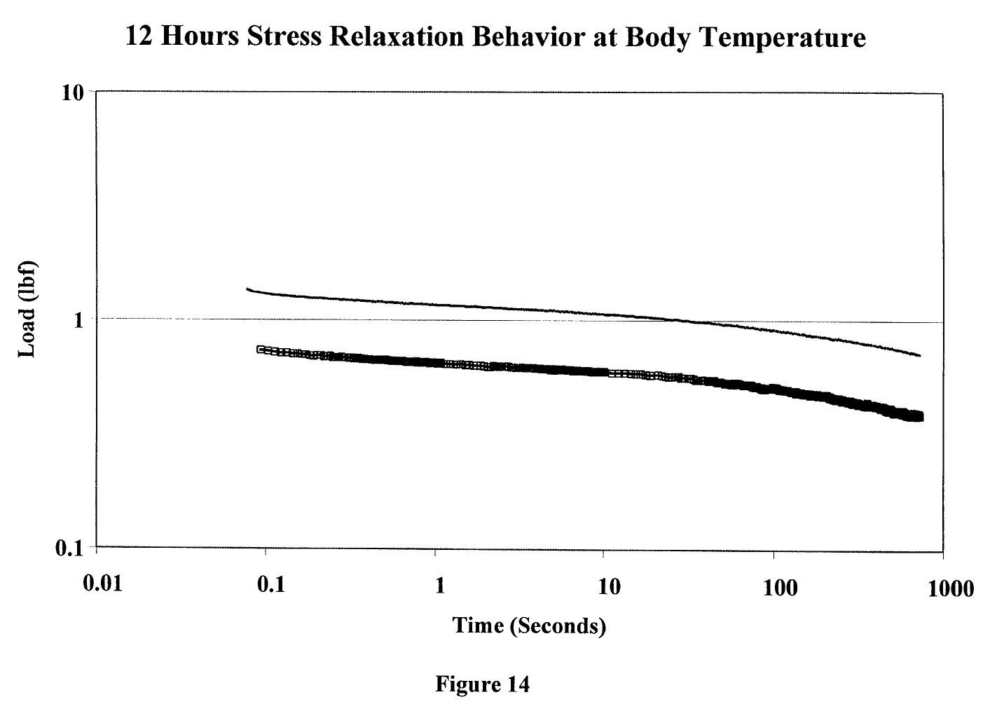

FIG. 14 shows the stress relaxation behavior of the TE and non-TE portions of the laminate. Table 1, below, shows the load decay rates and load loss at the end of 12 hours for the TE and non-TE materials. LYCRA® spandex was included as a control.

TABLE 1

| Laminate | Load Decay Rate | 1% Load Loss (12 hr) |
|---|---|---|
| Control (CFSBL) | −0.08 | 50 |
| TE Zone | −0.07 | 48 |
| Non-TE Zone | −0.08 | 49 |
| LYCRA ® spandex | −0.02 | 10 |

3-Cycle Hysteresis Test

Equilibrium hysteresis behavior of the polymers was obtained by ramping a rectangular specimen up to 160% and down to 0% elongation at 20 inches/minute at room temperature. The procedure was repeated 3 times. Most of the samples attained equilibrium in 2 to 3 up-and-down ramping cycles.

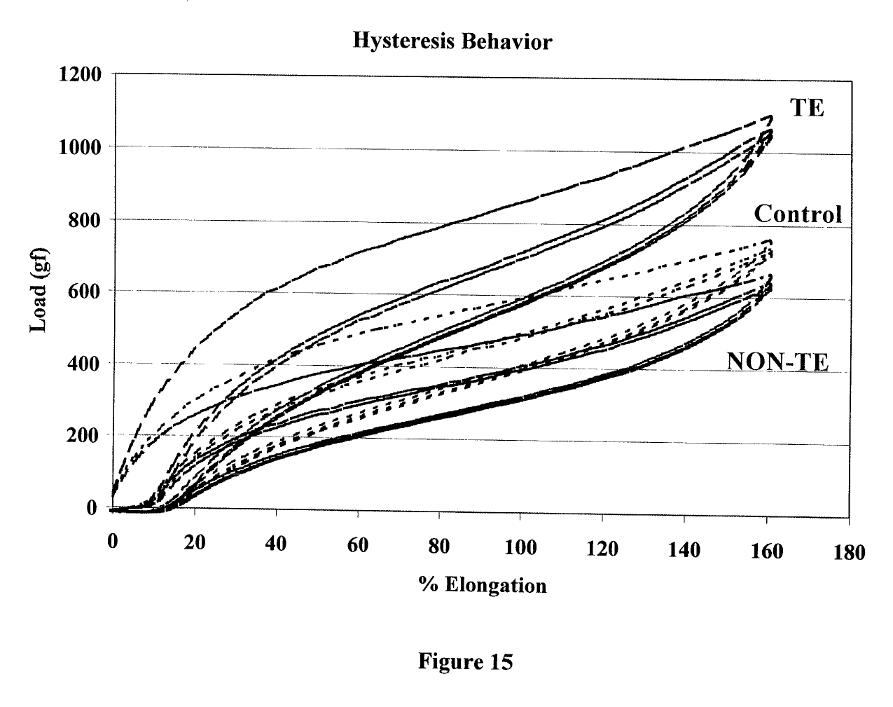

The three curves shown in FIG. 15 are for the targeted high-tension TEM, the control (PULL-UPS® Disposable Training Pants with uniform tension), and the low tension targeted elastic laminates. The curves also serve the purpose of illustrating the donning process to which a product might be subjected before putting the product on the user. It can be seen from the figure that each material loses some of its tension on the second and third loading in comparison with the first loading cycle. However, the tension remains relatively constant for all three unloading cycles. The second and third loading cycles have similar loading tensions as a function of elongation. It can also be seen that in all cases some of the lost load on unloading is restored on the loading cycles. The figure illustrates that the tension of the control is in between the targeted and non-targeted elastic materials.

Stress Elongation

The stress-elongation behavior of the laminates was obtained at room temperature using a Sintech 1/S testing frames. Rectangular laminate samples having 3-inch widths were clamped at a grip-to-grip distance of 3 inches and were pulled at a cross-head displacement of 20 inches/minute. Samples were stretched to approximately 2000 grams load limit. The elongation was calculated from knowledge of the change in length and the original length of the sample. The tension at 50% elongation was calculated from the data acquired.

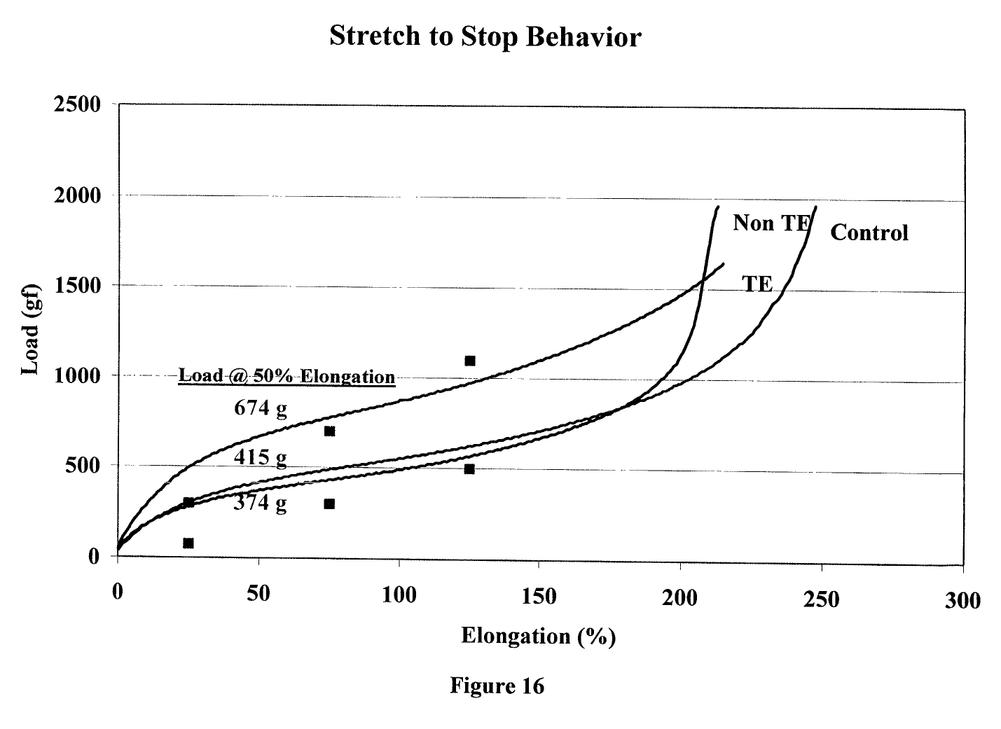

FIG. 16 shows the stress elongation curves for the TE, non-TE and control laminate samples. The TE portion was a 2″ wide film made up of 65.5% KRATON® G1730, 12% of a low molecular weight polyethylene wax, NA 601, and 22.5% of a pressure sensitive adhesive such as Regalrez®, on top of strands of 85% KRATON® G1730 and 15% wax, of less than 0.03 inch diameter at 12 strands per inch. The number of strands per inch and the thickness of the TE film can be changed independently or in combination, to alter the load-elongation characteristics of the elastic laminate material. The 3-inch samples tested had 1 to 2.5-inch wide film and elastic strand overlaid on it. The additional 0.5 to 2 inches of material consisted of the non-TE portion. In other words, TE samples tested had a width of 3 inches consisting of both TE and non-TE portions. The TE and non-TE portions could also be tested separately to define the material specifications. It can be seen from FIG. 16 that the tension as a function of elongation is lower (up to about 150%) for the non-TE portions and higher for the TE portions. The TE panel also provides an additional advantage. Having a higher tension as a function of elongation of the side panel material means that when the TE panel stress decays as a function of time at body temperature, it will still be at a higher tension than the control and non-TE material after a given period of time. For example, consider the TE material, which has 674 grams at 50% elongation. Examination of Table 1 shows that this material stress relaxes 50% in 12 hours at body temperature. This implies that after 12 hours the material will be at a load of 324 grams. Compare this value with the control, which is at 415 grams at 50% elongation and it stress relaxes 50% after 12 hours. Fifty percent of 415 is 208 grams. Thus the TE material is at 116 grams higher than the control at the end of 12 hours which delivers better tension to the body and therefore better body fit over time.

The invention further encompasses various types of garments in which a high tension and/or low stretch gasketing elastic zone is present in the vicinity of any one or more garment openings. Depending on the garment, high tension and/or low stretch gasketing zones of a TEM may encircle an entire garment opening or just a portion of the garment opening. In addition to the training pant 20, other types of garments on which this invention can be used include personal care garments, such as diapers, absorbent underpants, adult incontinence products, certain feminine hygiene articles, and swim wear. The high tension and/or low stretch gasketing elastic zones may be used in similar fashion in medical garments including, for instance, medical gowns, caps, gloves, drapes, face masks, and the like, where it is desired to provide a gasket in the vicinity of one or more garment openings without requiring a separately manufactured and attached elastic band. Furthermore, the high tension and/or low stretch gasketing elastic zone can be used around neck openings, arm openings, wrist openings, waist openings, leg openings, ankle openings, and any other opening surrounding a body part wherein fluid transfer resistance is desirable.

While the embodiments of the invention described herein are presently preferred, various modifications and improvements can be made without departing from the spirit and scope of the invention. The scope of the invention is indicated in the appended claims, and all changes that fall within the meaning and range of equivalents are intended to be embraced therein.

What is claimed:

1. An elastomeric laminate, comprising:
a. an elastomeric film having a first major surface and a second major surface; and
b. a strand of an elastomeric material secured to the first major surface of the elastomeric film.

2. The elastomeric laminate according to claim 1, wherein:
the elastomeric strand material comprises a thermoplastic polymer.

3. The elastomeric laminate according to claim 1, wherein:
the elastomeric film material comprises a thermoplastic polymer.

4. The elastomeric laminate according to claim 1, wherein:
the elastomeric strand material comprises a thermoset polymer.

5. The elastomeric laminate according to claim 1, wherein:
the elastomeric film material comprises a thermoset polymer.

6. The elastomeric laminate according to claim 1, wherein:
the elastomeric composition of the strand material is the same as the elastomeric composition of the film material.

7. The elastomeric laminate according to claim 1, wherein:
the elastomeric composition of the strand material is different than the elastomeric composition of the film material.

8. The elastomeric laminate according to claim 1, further comprising:
a facing sheet bonded to the laminate.

9. The elastomeric laminate according to claim 8, wherein:
both major surfaces of the laminate are covered with facing sheets.

10. The elastomeric laminate according to claim 8, wherein:
the facing sheet is a spunbond sheet.

11. The elastomeric laminate according to claim 8, further comprising:
a garment incorporating the elastomeric laminate and the facing sheet into the structure of the garment.

12. The elastomeric laminate according to claim 11 wherein the garment is one selected from the group of personal care garments, medical garments and industrial workwear garments.

13. The elastomeric laminate according to claim 12 wherein the garment is one selected from the group of: diapers, training pants, swim wear, absorbent underpants, adult incontinence products, feminine hygiene products, protective medical gowns, surgical medical gowns, caps, gloves, drapes, face masks laboratory coats and coveralls.

14. The elastomeric laminate according to claim 1, further comprising:
a garment incorporating the elastomeric laminate into the structure of the garment.

15. The elastomeric laminate according to claim 1, wherein:
different portions of the elastomeric film exhibit different amounts of elastic tension.

16. The elastomeric laminate according to claim 15, further comprising a plurality of elastomeric strands on the elastomeric film surface, wherein at least some of the elastomeric strands exhibit different amounts of elastic tension.

17. The elastomeric laminate according to claim 1, wherein:
there are multiple elastomeric strands on the elastomeric film surface.

18. The elastomeric laminate according to claim 17, wherein:
at least some of the elastomeric strands exhibit different amounts of elastic tension.

19. The elastomeric laminate according to claim 17, wherein:
at least some of the elastomeric strands have different thicknesses.

20. The elastomeric laminate according to claim 17, wherein:
at least some of the elastomeric strands have different compositions.

21. The elastomeric laminate according to claim 17, wherein:
the elastomeric strands are arranged in periodic spacing.

22. The elastomeric laminate according to claim 17, wherein:
the elastomeric strands are arranged in nonperiodic spacing.

23. The elastomeric laminate according to claim 17, wherein:
the elastomeric strands are arranged in groups.

24. The elastomeric laminate according to claim 23, wherein:
at least some of the groups exhibit different amounts of elastic tension from each other.

25. The elastomeric laminate according to claim 23, wherein:
the groups have different spacing between their elastomeric strands.

26. The elastomeric laminate according to claim 1, further comprising:
a second elastomeric strand secured to the second major surface of the elastomeric film.

27. The elastomeric laminate according to claim 1, wherein:
a second elastomeric film contacts the elastomeric strand thereby placing the elastomeric strand between the elastomeric film and the second elastomeric film.

28. An elastomeric laminate, comprising:
a. an elastomeric film having a first major surface and a second major surface;
b. a plurality of strands of an elastomeric polymer material secured to the first major surface of the elastomeric film; and
c. a facing sheet bonded to at least one of the elastomeric film or the elastomeric strands.

29. A process of making an elastomeric laminate, comprising:
a. producing an elastomeric film;
b. producing elastomeric strands;
c. securing the elastomeric strands to the elastomeric film.

30. The process of making an elastomeric laminate according to claim 29, further comprising: the step of producing elastomeric film including placing elastomeric material extruded from a slotted film die onto a cooling roll; and stretching the elastomeric film from the cooling roll towards a nip formed between two nip rollers.

31. The process of making an elastomeric laminate according to claim 30, further comprising: adding a tackifier to a formulation of the film.

32. The process of making an elastomeric laminate according to claim 30, further comprising: the step of producing elastomeric strands including placing elastomeric material extruded from a filament die onto a cooling roll; and stretching the elastomeric strands from a cooling roll towards the nip formed between two nip rollers.

33. The process of making an elastomeric laminate according to claim 32, further comprising: securing the elastomeric film and the elastomeric strands together in the nip.

34. A garment having an elastomeric laminate therein which is made by the process of claim 33.

35. The process of making an elastomeric laminate according to claim 29, further comprising: the step of producing elastomeric strands including placing elastomeric material extruded from a filament die onto a cooling roll; and stretching the elastomeric strands from a cooling roll towards a nip formed between two nip rollers.

36. The process of making an elastomeric laminate according to claim 35, further comprising: adding a tackifier to a formulation of the strands.

37. The process of making an elastomeric laminate according to claim 35 further including vertically stretching the elastomeric strands.

38. A garment having an elastomeric laminate therein which is made by the process of claim 35.

39. The process of making an elastomeric laminate according to claim 29, further comprising: spraying at least one of the film and strands with an adhesive before securement of the film and strands together.

40. The process of making an elastomeric laminate according to claim 29, wherein the film is extruded onto a first cooling roll and the strands are extruded onto a second cooling roll.

41. The process of making an elastomeric laminate according to claim 29, further including the step of keeping the laminate under tension with a pair of opposed tensioning rollers after it passes through the nip.

42. The process of making an elastomeric laminate according to claim 41 including relaxing the laminate after passage through the opposed tensioning rollers.

43. A garment having an elastomeric laminate therein which is made by the process of claim 41.

44. The process of making an elastomeric laminate according to claim 29, further including the step of adhering a facing sheet to one side of the laminate.

45. The process of making an elastomeric laminate according to claim 44, further including the step of adhering facing sheets onto both sides of the laminate.

46. The process of making an elastomeric laminate according to claim 44, further including the step of adhering the facing sheet to the laminate in the nip.

47. The process of making an elastomeric laminate according to claim 29, wherein at least one of the elastomeric film and the elastomeric strands is a thermoset polymer that is cross-linked prior to securing the elastomeric strands to the elastomeric film.

48. The process of making an elastomeric laminate according to claim 29, wherein at least one of the elastomeric film and the elastomeric strands can be cross-linked after securing the elastomeric strands to the elastomeric film.

49. A garment having an elastomeric laminate therein which is made by the process of claim 29.

50. A process of making an elastomeric laminate, comprising:
a. extruding an elastomeric film onto a first cooling roll;
b. extruding elastomeric strands onto a second cooling roll;
c. feeding the elastomeric film and elastomeric strands from the first and second cooling rolls into a roller nip and securing the elastomeric strands to the elastomeric film;
d. adhering a facing sheet to at least one of the elastomeric film and elastomeric strands in the roller nip.
